SEQUENCE LISTING (24 sequences)

SEQ ID NO: 1 - DNA, 13 bp, synthetic construct
  source 1..13: mol_type = other DNA
ggcagctgttgct

SEQ ID NO: 2 - DNA, 771 bp, synthetic construct
  source 1..771: mol_type = other DNA
ccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcc

SEQ ID NO: 3 - DNA, 797 bp, synthetic construct
  source 1..797: mol_type = other DNA
ggcagctgttgctccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtcggcagctgttgcttagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcc

SEQ ID NO: 4 - DNA, 823 bp, synthetic construct
  source 1..823: mol_type = other DNA
ggcagctgttgctggcagctgttgctccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtcggcagctgttgctggcagctgttgcttagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcc

SEQ ID NO: 5 - DNA, 888 bp, synthetic construct
  source 1..888: mol_type = other DNA
ggcagctgttgctggcagctgttgctggcagctgttgctccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtcggcagctgttgctggcagctgttgctggcagctgttgcttagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaaggcagctgttgctggcagctgttgctggcagctgttgctcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcc

SEQ ID NO: 6 - DNA, 53 bp, synthetic construct
  source 1..53: mol_type = other DNA
ttcacgcgtggcagctgttgctccttcagattaaaaataactgaggtaagggc

SEQ ID NO: 7 - DNA, 46 bp, synthetic construct
  source 1..46: mol_type = other DNA
gcggccattcggtacaattcacgcgtggcagctgttgctccttcag

SEQ ID NO: 8 - DNA, 37 bp, synthetic construct
  source 1..37: mol_type = other DNA
aagcaacagctgccgacagcagggccccaaggtttgc

SEQ ID NO: 9 - DNA, 50 bp, synthetic construct
  source 1..50: mol_type = other DNA
ggccctgctgtcggcagctgttgcttagcatgccccactacgggtctagg

SEQ ID NO: 10 - DNA, 43 bp, synthetic construct
  source 1..43: mol_type = other DNA
ggtggcgaccggtggatcggccgcggctggctggctcctgagt

SEQ ID NO: 11 - DNA, 49 bp, synthetic construct
  source 1..49: mol_type = other DNA
ggccgatccaccggtcgccaccatggaagatgccaaaaacattaagaag

SEQ ID NO: 12 - DNA, 46 bp, synthetic construct
  source 1..46: mol_type = other DNA
ggttgattatctcgagcggaattctcatcgagctcgagatctggcg

SEQ ID NO: 13 - DNA, 58 bp, synthetic construct
  source 1..58: mol_type = other DNA
gtggcagctgttgctggcagctgttgctccttcagattaaaaataactgaggtaaggg

SEQ ID NO: 14 - DNA, 58 bp, synthetic construct
  source 1..58: mol_type = other DNA
ccattcggtacaattcacgcgtggcagctgttgctggcagctgttgctccttcagatt

SEQ ID NO: 15 - DNA, 59 bp, synthetic construct
  source 1..59: mol_type = other DNA
ggggcatgctaagcaacagctgccagcaacagctgccgacagcagggccccaaggtttg

SEQ ID NO: 16 - DNA, 55 bp, synthetic construct
  source 1..55: mol_type = other DNA
gtcggcagctgttgctggcagctgttgcttagcatgccccactacgggtctaggc

SEQ ID NO: 17 - DNA, 57 bp, synthetic construct
  source 1..57: mol_type = other DNA
ctgttgctggcagctgttgctccttcagattaaaaataactgaggtaagggcctggg

SEQ ID NO: 18 - DNA, 67 bp, synthetic construct
  source 1..67: mol_type = other DNA
cattcggtacaattcacgcgtggcagctgttgctggcagctgttgctggcagctgttgctccttcag

SEQ ID NO: 19 - DNA, 33 bp, synthetic construct
  source 1..33: mol_type = other DNA
gttcctgcggccattcggtacaattcacgcgtg

SEQ ID NO: 20 - DNA, 59 bp, synthetic construct
  source 1..59: mol_type = other DNA
caacagctgccagcaacagctgccagcaacagctgccgacagcagggccccaaggtttg

SEQ ID NO: 21 - DNA, 59 bp, synthetic construct
  source 1..59: mol_type = other DNA
tggggcatgctaagcaacagctgccagcaacagctgccagcaacagctgccgacagcag

SEQ ID NO: 22 - DNA, 40 bp, synthetic construct
  source 1..40: mol_type = other DNA
gcagctgttgcttagcatgccccactacgggtctaggctg

SEQ ID NO: 23 - DNA, 59 bp, synthetic construct
  source 1..59: mol_type = other DNA
gcaacagctgccagcaacagctgccagcaacagctgccttcgaagatcttcgcatgcag

SEQ ID NO: 24 - DNA, 59 bp, synthetic construct
  source 1..59: mol_type = other DNA
ggcagctgttgctggcagctgttgctggcagctgttgctcaaggctgtgggggactgag

(19) United States
(12) Patent Application Publication
(10) Pub. No.: US 2026/0014280 A1
(43) Pub. Date: Jan. 15, 2026

(54) PROMOTER SEQUENCE FOR INITIATING GENE SPECIFICALLY IN MAMMALIAN MUSCLE AND USE THEREOF

(71) Applicant: PACKGENE BIOTECH CO., LTD., Guangzhou (CN)

(72) Inventors: Ye Bu, Guangzhou (CN); Huapeng Li, Guangzhou (CN); Junlin Chen, Guangzhou (CN); Keqin Tan, Guangzhou (CN); Yujian Zhong, Guangzhou (CN); Yue Pan, Guangzhou (CN); Huan Chen, Guangzhou (CN)

(21) Appl. No.: 19/251,849

(22) Filed: Jun. 27, 2025

Related U.S. Application Data
(63) Continuation of application No. PCT/CN2023/072584, filed on Jan. 17, 2023.

(30) Foreign Application Priority Data
Dec. 30, 2022 (CN) 202211731527.2

(51) Int. Cl. A61K 48/00; C12N 15/86

(52) U.S. Cl. CPC A61K 48/0058; C12N 15/86; C12N 2750/14143; C12N 2830/008

(57) ABSTRACT

The present disclosure provides a muscle-specific chimeric promoter, including: (1) a hybrid α-myosin heavy chain enhancer/muscle creatine kinase enhancer-promoter (MHCK7 promoter); and (2) one or more binding sites of a transcription factor, wherein the MHCK7 promoter includes the sequence set forth in SEQ ID NO: 2 or a functional variant thereof having at least 90% sequence identity to SEQ ID NO: 2, and wherein the transcription factor is a member selected from the group consisting of the MyoD family of transcription factors. Compared with the MHCK7 promoter, the specificity and expression ability in muscle tissue of the muscle-specific chimeric promoter provided in the present application are significantly improved. The application of the muscle-specific chimeric promoter in mammals and muscle cells is of great significance for the treatment of muscle diseases.

### CROSS-REFERENCE TO RELATED APPLICATIONS

The present application is a Continuation Application of PCT application No. PCT/CN2023/072584 filed on Jan. 17, 2023, which claims the benefit of Chinese Patent Application No. 202211731527.2 filed on Dec. 30, 2022. The contents of the above-identified applications are hereby incorporated by reference.

### REFERENCE TO SEQUENCE LISTING

This application includes a Sequence Listing filed electronically as an XML file named “U.S. Pat. No. 2,401,293H-PCT_SL.xml”, created on May 9, 2025, with a size of 26,030 bytes. The Sequence Listing is incorporated herein by reference.

### TECHNICAL FIELD

The present disclosure relates to a promoter sequence and a use thereof, and particularly a promoter sequence capable of driving the expression of a target gene (or initiating transcription of a target gene) specifically in muscle tissue and a use thereof.

### BACKGROUND

There is a lack of effective treatments for hereditary muscle diseases, which often result in high morbidity and mortality due to skeletal muscular and cardiac dysfunction. Gene therapy is an effective and promising treatment for genetic diseases and is in a high growth phase. Gene therapy offers hope for many genetic disorders, including muscle diseases. Viral vectors (including vectors such as lentiviruses and adeno-associated viruses) are commonly used for delivery of genes in gene therapy. However, achieving efficient gene delivery in muscle cells is still challenging at present. It is an effective way to improve the application of viral vectors in gene therapy for hereditary muscle diseases by developing muscle tissue-specific promoters to enhance the specific tissue expression of viral vector drugs, reduce the non-specific expression of delivery genes, achieve controllable drug release from viral vectors and reduce the usage amount of the viral vectors.

The hybrid α-myosin heavy chain enhancer/muscle creatine kinase enhancer-promoter (MHCK7 promoter) was obtained by Salva M Z et al.^{[1]} via optimizing a muscle creatine kinase (CK) promoter in mice. The MHCK7 promoter has good expression efficiency and specificity in muscle tissues of mouse and primate, and has been widely used in research and preclinical experiments^{[2]}. However, the MHCK7 promoter still has certain shortcomings, such as the expression efficiency needs to be further improved. The MyoD1 transcription factor (Myoblast determination protein 1) acts as a transcriptional activator, promotes transcription of muscle-specific target genes, and plays a role in muscle differentiation^{[3]}. In this application, a recognition site of the MyoD1 transcription factor is added to the MHCK7 promoter, so as to optimize the MHCK7 promoter and obtain good effects.

### SUMMARY

In one aspect, the present application provides a muscle-specific chimeric promoter (chimeric promoter having muscle specificity), including: (1) an MHCK7 promoter; and (2) one or more binding sites of a transcription factor (transcription factor binding site, TFBS), wherein the MHCK7 promoter includes the sequence set forth in SEQ ID NO: 2 or a functional variant thereof having at least 90% sequence identity to SEQ ID NO: 2, and wherein the transcription factor is a member selected from the group consisting of the MyoD family of transcription factors.

In some embodiments, the transcription factor is MyoD1 and/or Myog.

In some embodiments, the number of the one or more binding sites of the transcription factor is 2-9.

In some embodiments, the number of the one or more binding sites of the transcription factor is 2, 4, or 9.

In some embodiments, the one or more binding sites of the transcription factor include the sequence set forth in SEQ ID NO: 1, or a functional variant comprising 1 or 2 nucleotide changes when compared to the sequence set forth in SEQ ID NO: 1.

In some embodiments, the one or more binding sites of the transcription factor are located upstream and/or downstream of the MHCK7 promoter and/or located within the MHCK7 promoter.

In some embodiments, the muscle-specific chimeric promoter includes the sequence set forth in SEQ ID NO: 3, SEQ ID NO: 4, or SEQ ID NO: 5, or a functional variant having 90% sequence identity to any one of SEQ ID NOs: 3-5.

In some embodiments, the muscle-specific chimeric promoter has a higher capacity to initiate transcription in muscle tissue when compared to the MHCK7 promoter.

In some embodiments, the muscle tissue is selected from the group consisting of skeletal muscle and cardiac muscle; preferably, the skeletal muscle is a bicep (biceps brachii) and/or a quadricep (quadriceps femoris).

In another aspect, the present application provides a gene expression cassette including the muscle-specific chimeric promoter above and a target gene operably linked to the muscle-specific chimeric promoter.

In another aspect, the present application provides an expression vector including the muscle-specific chimeric promoter above or the gene expression cassette above.

In some embodiments, the expression vector is a viral expression vector.

In some embodiments, the expression vector is an expression vector of an adeno-associated virus (AAV).

In another aspect, the present application provides a host cell including the muscle-specific chimeric promoter, the gene expression cassette, or the expression vector above.

In another aspect, the present application provides a pharmaceutical composition, including: (1) the gene expression cassette, the expression vector, or the host cell above; and (2) a pharmaceutically acceptable carrier.

In another aspect, the present application provides the use of the above-mentioned gene expression cassette, expression vector, host cell, or pharmaceutical composition in the preparation of a drug for the treatment of a muscle tissue-related disease.

In another aspect, the present application provides a method of treating a muscle tissue-related disease, including administering to a subject in need an effective amount of the gene expression cassette, the expression vector, the host cell, or the pharmaceutical composition above.

The MHCK7 promoter is optimized by adding different numbers of sequences of a recognition binding site of the MyoD1 transcription factor at specific locations. Compared with the unoptimized MHCK7 promoter, the optimized promoter has good muscle tissue specificity, the transcriptional activity in muscle tissue has been greatly improved, and the expression of the target protein in muscle tissue has been significantly increased (for example, the expression level has increased by 2.63-4.45 times), as verified by in vivo mouse experiments.

### BRIEF DESCRIPTION OF THE DRAWINGS

FIG. 1 shows fluorescence images of mouse in vivo imaging when different promoters were used. Group A: pAAV.MHCK7.Fluc-2a-maxGFP.WPRE.SV40pA; group B: pAAV.MHCK7-1.Fluc-2a-maxGFP.WPRE.SV40pA; group C: pAAV.MHCK7-2.Fluc-2a-maxGFP.WPRE.SV40pA; group D: pAAV.MHCK7-3.Fluc-2a-maxGFP.WPRE.SV40pA; and the exposure time was 200 ms.

FIG. 2A, FIG. 2B, FIG. 2C, FIG. 2D, FIG. 2E, FIG. 2F, and FIG. 2G show the expression levels of luciferace mRNA in different tissues determined by qPCR. FIG. 2A shows the results of qPCR determination of luciferase gene expression in different tissues using different promoters. FIG. 2B shows the results of luciferase gene expression in heart tissues. FIG. 2C shows the results of luciferase gene expression in biceps. FIG. 2D shows the results of luciferase gene expression in quadriceps. FIG. 2E shows the results of luciferase gene expression in liver tissues. FIG. 2F shows the results of luciferase gene expression in lung tissues. FIG. 2G shows the results of luciferase gene expression in brain tissues. Group A: pAAV.MHCK7.Fluc-2a-maxGFP.WPRE.SV40Pa; group B: pAAV.MHCK7-1.Fluc-2a-maxGFP.WPRE.SV40pA; group C: pAAV.MHCK7-2.Fluc-2a-maxGFP.WPRE.SV40pA; D: group pAAV.MHCK7-3.Fluc-2a-maxGFP.WPRE.SV40pA; and group A was used as the control. Student's t-test was performed for statistical analysis. Among FIGS. 2B-2G, * represents p<0.05,** represents p<0.01, and ns represents no significant difference.

FIG. 3A and FIG. 3B show the photos and grayscale analysis results of luciferase expression detected by a western blot. FIG. 3A shows the results in biceps; and FIG. 3B shows the results in quadriceps. Group A: pAAV.MHCK7.Fluc-2a-maxGFP.WPRE.SV40pA; group C: pAAV.MHCK7-2.Fluc-2a-maxGFP.WPRE.SV40pA; and grayscale analysis was performed using group A as the control. Student's t-test was performed for statistical analysis. Among them, ** represents p<0.01.

### DETAILED DESCRIPTION

Unless otherwise indicated, all technical and scientific terms used herein have the meanings commonly understood by those of ordinary skill in the art.

The term “or” refers to a single element of the enumerated optional elements, unless the context clearly indicates otherwise. The term “and/or” refers to any one, any two, any three, any more or all of the optional elements listed.

The terms “comprises”, “includes”, “has” and similar expressions are used herein to indicate that elements not enumerated are not excluded. These terms also include situations that consist only of the enumerated elements.

The term “promoter” refers to a DNA sequence where RNA polymerase recognizes, binds to, and initiates transcription. It contains a conserved sequence required for RNA polymerase-specific binding and transcription initiation. Most promoters are located upstream (in the 5′ direction) of the transcription start site of structural genes. The promoter itself is not transcribed. Examples of promoters include, but are not limited to, cytomegalovirus (CMV) promoter, elongation factor 1 alpha (EF1A) promoter, cytomegalovirus enhancer/chicken beta-actin (CAG) promoter, cytomegalovirus enhancer/chicken beta actin hybrid (CBh) promoter, spleen focus forming virus (SFFV) promoter and the like.

The term “chimeric promoter” may also be called a “combined promoter” or “composite promoter”. The “chimeric promoter” refers to a promoter that, in addition to the promoter sequence, also includes at least one transcriptional regulatory element, and the transcriptional regulatory clement and the promoter are not naturally present in the transcriptional regulatory sequence of the same gene. For example, the promoter naturally exists in the transcriptional regulatory sequence of a first gene, and another transcriptional regulatory element (such as a recognition binding site of a transcription factor) naturally exists in the transcriptional regulatory sequence of a second gene; when these two transcriptional regulatory elements are artificially manipulated to be in the same DNA molecule and control the transcription of the same gene, they can be considered to constitute a chimeric promoter. In addition, an additional transcriptional regulatory element can be further added to the chimeric promoter to form a new chimeric promoter. In this case, the basic chimeric promoter may be directly referred to as a promoter in order to distinguish the basic chimeric promoter from the new chimeric promoter.

When referring to a chimeric promoter or other transcriptional regulatory elements, “muscle-specific” (or muscle specificity) means that the chimeric promoter or other transcriptional regulatory elements preferentially drive or enhance the expression of an operably linked target gene in muscle tissue (e.g., skeletal muscle or cardiac muscle). “Muscle specificity” (or muscle-specific) does not exclude the possibility that the chimeric promoter or other transcriptional regulatory elements drive or enhance the expression of the operably linked target gene to some extent in another tissue, only that its expression is lower relative to that in muscle. For example, a muscle-specific chimeric promoter (chimeric promoter having muscle specificity) can drive the expression of a target gene in both muscle and liver tissues. However, the expression level of the target gene in muscle is more than 2 times, or more than 5 times, or more than 10 times, or higher than the expression level in liver tissue.

The term “transcriptional regulatory element” refers to a nucleotide fragment that is capable of driving (e.g., a promoter) or enhancing (e.g., an enhancer) the expression of an operably linked target gene in a tissue or cell. The term “transcriptional regulatory sequence” refers to the sum of transcriptional regulatory elements that control the expression of a target gene, which may exist continuously or intermittently in the same DNA molecule.

The term “operably linked” refers to a regulatory sequence that is linked to its regulated object in such a way that the regulatory sequence can exert its effects on its regulatory object. For example, a promoter is “operably linked” to a target gene, which means that the promoter can drive transcription of the target gene from a precise start site.

The term “binding sites of a transcription factor” or “transcription factor binding site” (TFBS) refers to a nucleotide sequence on a DNA molecule that a transcription factor can recognize and bind to. After the transcription factor binds to the “transcription factor binding site”, it helps to form a transcription initiation complex with other proteins (such as RNA polymerase) to initiate the transcription process.

The term “functional variant” or “functional fragment” refers to a protein or nucleic acid variant obtained after a minor modification (such as deletion, addition or substitution of an amino acid or nucleotide) based on the original sequence (such as a native sequence), which still retains all or at least part of the function of the original sequence. For example, a functional variant may retain 50%, 60%, 70%, 80%, 90%, 100% of a certain activity of the original sequence or even have an activity higher than that of the original sequence.

The term “MHCK7 promoter” herein refers to a promoter that is capable of driving the expression of a target gene in muscle tissue. The MHCK7 promoter itself is a chimeric promoter, which includes the sequence set forth in SEQ ID NO: 2 or a functional variant thereof having at least 90% sequence identity to SEQ ID NO: 2.

As used herein, the terms “nucleic acid molecule”, “nucleic acid”, and “polynucleotide” are used interchangeably to refer to a polymer of nucleotides. Such nucleotide polymers may contain natural and/or non-natural nucleotides and include, but are not limited to, DNA, RNA, and peptide nucleic acid (PNA). The term “nucleic acid sequence” refers to a linear sequence of nucleotides contained in a nucleic acid molecule or polynucleotide. For DNA molecules, due to double-stranded complementarity, when referring to the sequence of one strand of DNA herein, those skilled in the art will be aware that the complementary strand or a double-stranded DNA molecule including the complementary strand is also referred to.

The term “vector” refers to a nucleic acid molecule that can be engineered to contain a polynucleotide of interest (e.g., a coding sequence for a polypeptide of interest) or a nucleic acid molecule that can replicate in a host cell (e.g., a nucleic acid, a plasmid, or a virus, etc.). The vector may include one or more of the following components: an origin of replication, one or more regulatory sequences (such as a promoter and/or enhancer) that regulate the expression of the polynucleotide of interest, and/or one or more selectable marker genes (such as antibiotic resistance genes and genes that can be used in colorimetric assays, e.g., β-galactose). The term “expression vector” refers to a vector used to express a target gene in a host cell.

The term “host cell” refers to a cell that can be or has been a recipient of a vector or isolated polynucleotide. The host cell can be a prokaryotic cell or a eukaryotic cell. Exemplary eukaryotic cells include mammalian cells, such as primate or non-primate cells; fungal cells, such as yeast; plant cells; and insect cells. Non-limiting exemplary mammalian cells include, but are not limited to, NSO cells, 293 and CHO cells, and derivative cells thereof, such as 293-6E, CHO-DG44, CHO-K1, CHO-S, and CHO-DS cells. A host cell includes progeny of a single host cell, and the progeny may not necessarily be completely identical (in morphology or genomic DNA complement) to the original parent cell due to natural, accidental, or deliberate mutation. A host cell also includes a cell transfected in vivo with the nucleic acid molecule or an expression vector provided herein.

The term “target gene” means a polynucleotide sequence encoding an RNA or protein product. The polynucleotide sequence can be introduced into a cell or an individual according to the desired purpose and can be expressed under suitable conditions. The target gene may encode a product of interest, such as a therapeutic or diagnostic product of interest. A therapeutic target gene may be used, introduced into a cell, a tissue or an organ and expressed to produce the desired therapeutic outcome. Treatment can be achieved in a variety of ways, including by expressing a protein in cells that do not express the protein, by expressing a protein in cells that express a mutated version of the protein, by expressing a protein that is toxic to the target cells that express it (a strategy used, for example, to kill unwanted cells such as cancer cells), by expressing antisense RNAs to induce gene repression or exon skipping, or by expressing silencing RNAs, such as shRNAs, that are designed to inhibit protein expression. The target gene may also encode a nuclease for targeting the genome, such as a Clustered Regularly Interspaced Short Palindromic Repeats (CRISPR)-associated nucleic acid endonuclease or a transcription activator-like effector nuclease (TALEN). Alternatively, the target gene may be a guide RNA or a set of guide RNAs for use with the CRISPR/Cas9 system.

When referring to nucleotide sequences, the term “sequence identity” (also referred to as “sequence consistency”) refers to a measure of the degree of identity between two nucleotide sequences (e.g., a query sequence and a reference sequence), generally expressed as a percentage. Typically, a sequence comparison (alignment) is performed and a gap (if any) is introduced before calculating the percentage of identity between the two nucleotide sequences. If the bases in the two sequences are the same at a certain comparison position, the two sequences are considered to be consistent or matched at that position; if the bases in the two sequences are different, they are considered to be inconsistent or mismatched at that position. In some algorithms, the number of matching positions is divided by the total number of positions in the alignment window to obtain sequence identity. In other algorithms, the number of gaps and/or the length of the gaps are also taken into account. Commonly used sequence alignment algorithms or software include DANMAN, CLUSTALW, MAFFT, BLAST, MUSCLE, etc. For the purpose of the present disclosure, the publicly available alignment software BLAST (available from https://www.ncbi.nlm.nih.gov/) can be used to obtain the best sequence alignment and calculate the sequence identity between two nucleotide sequences by using the default settings.

The term “treatment” or “therapeutic” includes curative, palliative, or preventive effects. Thus, therapeutic and preventive treatments include improving the symptoms of the disorder or preventing or otherwise reducing the risk of developing specific symptoms. Treatment may be provided to delay, slow or reverse the progression of the disease and/or one or more of its symptoms. The term “prophylactic” can be thought of as reducing the severity or onset of a particular condition. The term “prophylactic” also encompasses the prevention of the recurrence of a condition in patients who have been previously diagnosed with a specific condition. The term “treatment” or “therapeutic” may also refer to the reduction of the severity of an existing condition. The term “treatment” is used herein to refer to any solution that may benefit an animal, particularly a mammal, and more particularly a human subject. In certain embodiments, the mammal may be a subject suffering from a disorder associated with the muscle tissue, such as a human patient.

### Chimeric Promoter

The inventors of the present application designed a transcriptional regulatory element referred to herein as a “chimeric promoter” for driving or enhancing the expression of a target gene in muscle tissue.

The chimeric promoter includes a MHCK7 promoter and one or more binding sites of a transcription factor (i.e., transcription factor binding site, TFBS), wherein the MHCK7 promoter includes the sequence set forth in SEQ ID NO: 2 or a functional variant thereof having at least 90% sequence identity to SEQ ID NO: 2, and wherein the transcription factor is a member selected from the group consisting of the MyoD family of transcription factors.

In the case where two or more transcription factor binding sites are present, these transcription factor binding sites (partial or all) can be directly connected in tandem or separated by a linker sequence. Direct tandem connection means that the first nucleotide of the latter transcription factor binding site immediately follows the last nucleotide of the upstream transcription factor binding site. In the case of connection via a linker sequence, other nucleotide sequences exist between the last nucleotide of the upstream transcription factor binding site and the first nucleotide of the subsequent downstream transcription factor binding site.

In some embodiments, where two or more transcription factor binding sites are present, the transcription factor binding sites may be selected from at least one of the group consisting of: a site located upstream of the MHCK7 promoter, a site located downstream of the MHCK7 promoter, and a site located within the MHCK7 promoter sequence. In some embodiments, when there are two or more transcription factor binding sites, the transcription factor binding sites are all located upstream or downstream of the MHCK7 promoter, or located within the MHCK7 promoter sequence. In other embodiments, when there are two or more transcription factor binding sites, the transcription factor binding sites are located upstream and downstream of the MHCK7 promoter, respectively. In other embodiments, when there are two or more transcription factor binding sites, the transcription factor binding sites are located upstream of the MHCK7 promoter, and located within the MHCK7 promoter, respectively. In other embodiments, when there are two or more transcription factor binding sites, the transcription factor binding sites are located downstream of the MHCK7 promoter, and located within the MHCK7 promoter, respectively. In other embodiments, when there are three or more transcription factor binding sites, the transcription factor binding sites are located upstream and downstream of the MHCK7 promoter, and located within the MHCK7 promoter, respectively. When referring to the positional relationship of two sequences in the same DNA molecule, the terms “upstream” and “downstream” mean that one sequence is located in the 5′ direction and 3′ direction of the other sequence, respectively. For example, sequence A is located upstream of sequence B, which means that sequence A is located in the 5′ direction of sequence B, and they may be directly connected or separated by other sequences. The “located within” refers to an internal position of a sequence where a specified sequence is inserted. For example, in some embodiments, when the MHCK7 promoter sequence includes SEQ ID NO: 2, “located within” may be the -581 base pair (bp)-582 bp position of SEQ ID NO: 2 and/or the -407-406 bp position of SEQ ID NO: 2. When the MHCK7 promoter sequence includes the functional variant of SEQ ID NO: 2 described in the present application, “located within” may be a position corresponding to the -581 bp--582 bp position of SEQ ID NO: 2 and/or the -407-406 bp position of SEQ ID NO: 2 in the functional variant.

In some embodiments, the number of the transcription factor binding sites is 2 or more, for example, 2-9, such as 2, 3, 4, 5, 6, 7, 8, 9 or more. In some embodiments, the number of the transcription factor binding sites is 2, 4, or 9.

In some embodiments, the transcription factor binding site may be a recognition binding site of a member of the MyoD family proteins. Members of the MyoD family proteins may include, for example, MyoD1, Myf5, MyoG, and Myf6 transcription factors. Preferably, the transcription factor binding site is the recognition binding site of the transcription factor MyoD1. More specifically, the transcription factor binding site includes the nucleotide sequence set forth in SEQ ID NO: 1.

In some preferred embodiments, the chimeric promoter includes the transcription factor binding site and the MHCK7 promoter in sequence from 5′ to 3′ direction, wherein the MHCK7 promoter additionally contains the incorporated transcription factor binding sites.

In addition, it is expected that the ability to express the target gene specifically in muscle may still be achieved by modifying (adding, replacing, or deleting) individual nucleotides of the above-mentioned transcriptional regulatory sequence (for example, a promoter comprising SEQ ID NO: 2 or a functional variant thereof, or a chimeric promoter comprising any one of SEQ ID NOs: 3-5). These modified functional variations should also be included in the scope of the present disclosure. For example, individual nucleotides (e.g., no more than 50, 20, 10, 5, 4, 3, 2 or 1 nucleotides) of the above-mentioned transcription regulatory sequence are altered (added, replaced or deleted), and their ability to activate the expression of the target gene is tested in vitro or in vivo to obtain functional variants of the muscle-specific chimeric promoter provided herein. These functional variants should also be included in the scope of the present disclosure. For example, in some embodiments, a functional variant may include a nucleotide sequence having at least 80%, 85%, 90%, 91%, 92%, 93%, 94%, 95%, 96%, 97%, 98%, 99% or even higher sequence identity to any of the above-mentioned transcriptional regulatory sequences.

The above-mentioned transcription regulatory elements can be directly connected or connected through a linker sequence. For example, the length of the linker sequence may be between 1 and 50 nucleotides, such as 1 to 40 nucleotides, such as 1 to 30 nucleotides, such as 1 to 20 nucleotides, such as 1 to 10 nucleotides. In some embodiments, the size constraints of the vector to be used may be taken into account in the design of the chimeric promoter. Therefore, such linker sequences, if present, are preferably short sequences. Representative short linker sequences include nucleic acid sequences consisting of less than 15 nucleotides, particularly less than 14, 13, 12, 11, 10, 9, 8, 7, 6, 5, 4, 3 or less than 2 nucleotides, such as a linker sequence of 1 nucleotide.

### Expression Cassette

The chimeric promoter provided herein can be introduced into an expression cassette designed to provide for expression of a target gene in a tissue of interest, such as muscle tissue.

Thus, the expression cassette provided herein includes the chimeric promoter and the target gene above.

In a specific embodiment, the expression cassette provided herein includes, from 5′ to 3′:
the chimeric promoter provided herein;
the target gene; and
a polyadenylation signal.

From the teachings disclosed herein and common knowledge in the fields of molecular biology and gene therapy, one skilled in the art may also consider incorporating other transcriptional regulatory elements into the chimeric promoter disclosed herein, such as introducing other enhancer sequences (e.g., a murine creatine kinase (MCK) enhancer or its functional variants) and intron sequences. The target gene that can be introduced for expression can include any gene of interest, especially therapeutic gene sequences associated with muscle disorders. These therapeutic genes are expected to be useful in the treatment of diseases such as muscular dystrophy (e.g., congenital muscular dystrophy), amyotrophic lateral sclerosis, inflammatory myopathies, muscle metabolic diseases (e.g., glycogen metabolism myopathy), myotonia congenita, and other neuromuscular disorders.

### Vector, Cell, and Pharmaceutical Composition

The expression cassettes provided herein can be introduced into a vector. Therefore, the present disclosure also relates to a vector including the expression cassette above. The vector used in the present disclosure is a vector suitable for RNA/protein expression, particularly suitable for gene therapy.

In some embodiments, the vector is a plasmid vector.

In other embodiments, the vector is a non-viral vector, such as a nanoparticle, lipid nanoparticle (LNP), or liposome containing an expression cassette of the present disclosure.

In other embodiments, the vector is a transposon-based system that allows integration of the expression cassette provided herein into the genome of the target cell.

In another embodiment, the vector is a viral vector suitable for gene therapy. In this case, as is known in the art, other sequences suitable for generating efficient viral vectors can be added to the expression cassette provided herein. In certain embodiments, the viral vector can be derived from an adenovirus, a retrovirus, or a lentivirus (e.g., an integration defective lentivirus). In the case where the viral vector is derived from a retrovirus or a lentivirus, the other sequences may be retroviral or lentiviral LTR sequences on both sides of the expression cassette. In another specific embodiment, the viral vector is a parvoviral vector, such as an AAV vector, such as an AAV vector suitable for transducing myocardium. In this embodiment, the other sequences are AAV inverted terminal repeat (ITR) sequences on either side of the expression cassette.

In a preferred embodiment, the vector is an AAV vector. Human adeno-associated virus (AAV) is a natural replication-defective Dependoparvovirus that is able to integrate into the genome of infected cells to establish a latent infection. AAV vectors have been widely used as vectors for human gene therapy. Advantageous properties of the viral vector include its lack of association with any human disease, its ability to infect both dividing and non-dividing cells, and the ability to infect a wide range of cell lines derived from different tissues.

Among the AAV serotypes isolated from humans or non-human primates (NHPs) and well characterized, human AAV serotype 2 was the first AAV to be developed as a gene transfer vector. Other AAV serotypes currently in use include AAV-1, AAV-3, AAV-4, AAV-5, AAV-6, AAV-7, AAV-8, AAV-9, AAV-10, etc. Additionally, other non-naturally occurring engineered variants and chimeric AAVs may also be useful.

AAVs can be engineered using conventional molecular biology techniques, such that these particles can be optimized for cell-specific delivery of nucleic acid sequences, for minimizing immunogenicity, for regulating stability and a particle lifespan, for efficient degradation, and for precise delivery to the cell nucleus.

The desired AAV segments for assembly into vectors include capsid proteins including vp1, vp2, vp3 and hypervariable regions, rep proteins including rep 78, rep 68, rep 52 and rep 40, and sequences encoding these proteins. These fragments can be readily utilized in a variety of vector systems and host cells.

The present disclosure also relates to an isolated cell, such as a muscle cell, which is transformed with a nucleic acid sequence of the present disclosure or an expression cassette of the present disclosure. The cells of the present disclosure can be delivered to a subject in need thereof by injection into a tissue of interest or into the bloodstream of the subject. In a specific embodiment, the present disclosure relates to introducing a nucleic acid molecule or an expression cassette of the present disclosure into cells of a subject to be treated, and administering the cells into which the nucleic acid or expression cassette has been introduced back to the subject.

Also provided herein is a pharmaceutical composition including the above-mentioned expression cassette, vector, or host cell. Such compositions include a therapeutically effective amount of the above-described expression cassette, vector, or cell, and a pharmaceutically acceptable carrier.

When referring to a pharmaceutical composition, the term “pharmaceutically acceptable carrier” refers to a solid or liquid diluent, filler, antioxidant, stabilizer, or other substances that can be safely administered. These substances are suitable for administration to a subject without undue adverse side effects, while being suitable for maintaining the activity of the drug or active agent located therein. Depending on the route of administration, various different carriers well known in the art can be used, including, but not limited to, sugars, starch, cellulose and its derivatives, maltose, gelatin, talc, calcium sulfate, vegetable oils, synthetic oils, polyols, alginic acid, phosphate buffer, emulsifiers, isotonic saline, and/or pyrogen-free water, etc.

If desired, the pharmaceutical composition may also contain small amounts of wetting agents or emulsifiers or pH buffering agents. These pharmaceutical compositions can take the form of solutions, suspensions, emulsions, tablets, pills, capsules, powders, sustained-release formulations and the like.

The pharmaceutical composition provided herein can be prepared into clinically acceptable dosage forms such as powders and injections. The pharmaceutical composition of the present disclosure can be administered to a subject using any appropriate route, for example, orally, intravenous infusion, intramuscular injection, subcutaneous injection, subperitoncally, rectally, sublingually, or by inhalation, transdermally, and the like.

In a preferred embodiment, the pharmaceutical composition is formulated according to routine procedures for intravenous or intramuscular administration. Typically, pharmaceutical compositions for intravenous or intramuscular administration are solutions in sterile isotonic aqueous buffer. If necessary, the pharmaceutical composition may also include a solubilizer and a local anesthetic such as lidocaine to relieve pain at the injection site of the subject.

As used herein, “subject” refers to an animal, such as a mammal, including but not limited to humans, rodents, apes and monkeys, felines, canines, equines, bovines, Suidae animals, sheep, goats, mammalian laboratory animals, mammalian farm animals, mammalian sport animals (e.g., race horses), and mammalian pets. The subject can be male or female and can be of any appropriate age, including infant, juvenile, adolescent, adult, and elderly subjects. In some examples, the subject is an individual in need of treatment for a disease or condition. In some examples, the subject receiving treatment may be a patient who suffers from a condition associated with the treatment, or is at risk of developing the condition. In certain examples, the subject is a human, such as a human patient. The term is often used interchangeably with “patient”, “subject”, “treatment subject”, and the like.

In one embodiment, the expression cassette or vector of the present disclosure may be delivered in a vesicle, in particular a liposome. In yet another embodiment, the nucleic acid sequence, expression cassette or vector of the present disclosure may be delivered in a controlled release system.

### Therapeutic Application

The chimeric promoter, expression cassette, or vector provided herein can be used to express a target gene in the myocardium. Therefore, in some embodiments, the present disclosure relates to the use of the above-mentioned expression cassette, vector, cell, or pharmaceutical composition in the preparation of a drug for treating myocardial related diseases.

The expression cassette and vector provided herein can also be used in gene therapy. Therefore, in some embodiments, the present disclosure relates to a method for treating a myocardium related disease, including administering an effective amount of the above-mentioned expression cassette, vector, cell, or pharmaceutical composition to a subject in need thereof.

In some embodiments, provided herein is a method for expressing a target gene in a muscle cell, including introducing an expression cassette or vector provided herein into the muscle cell, and expressing the target gene. The method may be an in vitro, ex vivo, or in vivo method for expressing a target gene in muscle cells.

In some embodiments, the present disclosure provides a method for expressing a target gene in myocardium, including introducing an expression cassette or expression vector provided herein into the myocardium, and expressing the target gene.

In certain embodiments, it may be desirable to administer the pharmaceutical composition of the present disclosure locally to an area in need of treatment, such as local myocardial tissue. This can be achieved, for example, using implants including porous, non-porous, or gel-like materials.

The dosage administered to a subject in need thereof will vary depending on several factors including, but not limited to, the route of administration, the specific disease being treated, the age of the subject, or the expression level necessary to achieve a therapeutic effect. Those skilled in the art can readily determine the required dosage according to these and other factors based on their knowledge in the art. In the case of administration with an AAV vector, a typical dosage of the vector is at least 1×10 ⁸ vector genomes per kilogram of body weight (vg/kg), such as at least 1×10 ⁹ vg/kg, at least 1×10 ¹⁰ vg/kg, at least 1×10 ¹¹ vg/kg, at least 1×10 ¹² vg/kg, at least 1×10 ¹³ vg/kg, at least 1×10 ¹⁴ vg/kg, or at least 1×10 ¹⁵ vg/kg. Of course, the physician may also select other dosages outside this range based on the individual condition of the subject.

The disclosure provides a promoter sequence for specifically initiating a gene in mammalian muscle and its application. The promoter sequence includes the nucleotide sequence of SEQ ID NO: 4 or a nucleotide sequence complementary thereto. The sequence is obtained by artificially optimizing a hybrid α-myosin heavy chain enhancer/muscle creatine kinase enhancer-promoter (MHCK7) sequence. Compared with the original promoter fragment, the specificity and expression ability in muscle tissue of the optimized promoter are significantly improved. The sequence involves applications in mammals and muscle cells, and has great significance for the treatment of muscle diseases.

The promoter sequences for specifically initiating genes in mammalian muscle provided herein and the annotations of various regulatory elements are shown in Table 1.

TABLE 1
Sequences of promoters and the transcription factor binding site

| Sequence name | Sequence (5′-3′) |
|---|---|
| The recognition and binding site | GGCAGCTGTTGCT (SEQ ID NO: 1) |
| of the MyoD1 transcription factor | |
| MHCK7 promoter sequence | CCTTCAGATTAAAAATAACTGAGGTAAGGGCCTGGGTAGG |
| | GGAGGTGGTGTGAGACGCTCCTGTCTCTCCTCTATCTGCCC |
| | ATCGGCCCTTTGGGGAGGAGGAATGTGCCCAAGGACTAAA |
| | AAAAGGCCATGGAGCCAGAGGGGCGAGGGCAACAGACCT |
| | TTCATGGGCAAACCTTGGGGCCCTGCTGTCTAGCATGCCCC |
| | ACTACGGGTCTAGGCTGCCCATGTAAGGAGGCAAGGCCTG |
| | GGGACACCCGAGATGCCTGGTTATAATTAACCCAGACATGT |
| | GGCTGCCCCCCCCCCCCCAACACCTGCTGCCTCTAAAAATA |
| | ACCCTGTCCCTGGTGGATCCCCTGCATGCGAAGATCTTCGA |
| | ACAAGGCTGTGGGGGACTGAGGGCAGGCTGTAACAGGCT |
| | TGGGGGCCAGGGCTTATACGTGCCTGGGACTCCCAAAGTAT |
| | TACTGTTCCATGTTCCCGGCGAAGGGCCAGCTGTCCCCCGC |
| | CAGCTAGACTCAGCACTTAGTTTAGGAACCAGTGAGCAAG |
| | TCAGCCCTTGGGGCAGCCCATACAAGGCCATGGGGCTGGG |
| | CAAGCTGCACGCCTGGGTCCGGGGTGGGCACGGTGCCCGG |
| | GCAACGAGCTGAAAGCTCATCTGCTCTCAGGGGCCCCTCC |
| | CTGGGGACAGCCCCTCCTGGCTAGTCACACCCTGTAGGCT |
| | CCTCTATATAACCCAGGGGCACAGGGGCTGCCCTCATTCTA |
| | CCACCACCTCCACAGCACAGACAGACACTCAGGAGCCAG |
| | CCAGCC (SEQ ID NO: 2) |
| Chimeric promoter 1 | 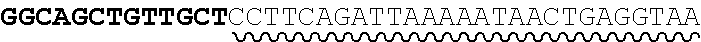 |
| (The sequences of recognition | 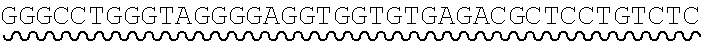 |
| and binding sites of the MyoD1 | 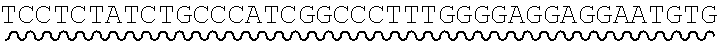 |
| transcription factor are shown in | 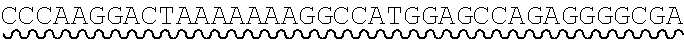 |
| bold, and the sequence contains | 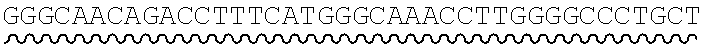 |
| two recognition sites of | 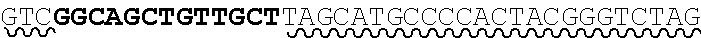 |
| “GGCAGCTGTTGCT” (SEQ | 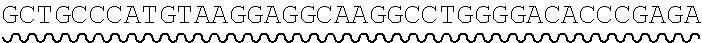 |
| ID NO: 1), which are located at | 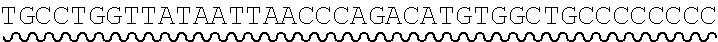 |
| the end of the MHCK7 promoter | 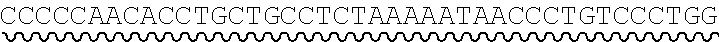 |
| sequence (i.e., at the -771 bp | 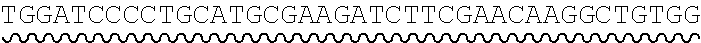 |
| position) and at the −581-−582 bp | 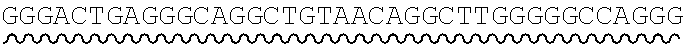 |
| position; and the underlined wavy | 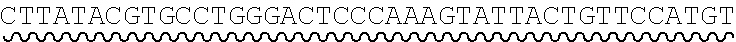 |
| line indicates the MHCK7 | 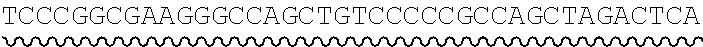 |
| promoter sequence.) | 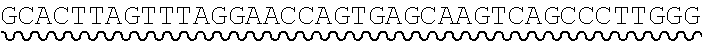 |
| | 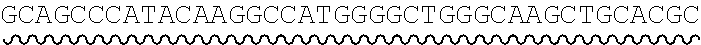 |
| | 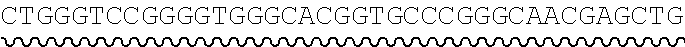 |
| | 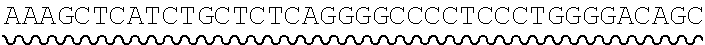 |
| | 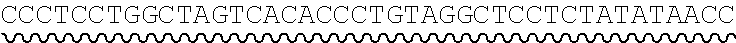 |
| | 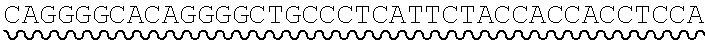 |
| | 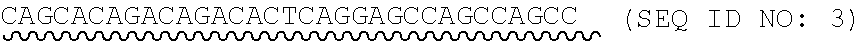 |
| Chimeric promoter 2 | 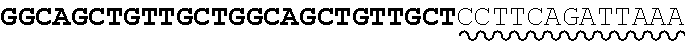 |
| (The sequences of recognition | 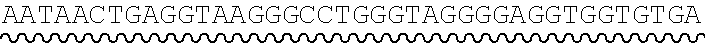 |
| and binding sites of the MyoD1 | 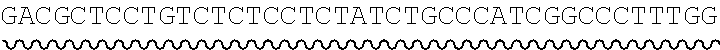 |
| transcription factor are shown in | 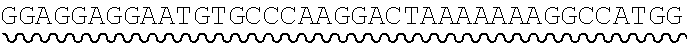 |
| bold, and the sequence contains | 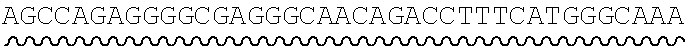 |
| two repeated recognition sites of | 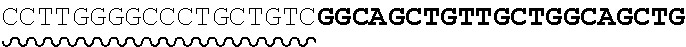 |
| “GGCAGCTGTTGCT” (SEQ ID | 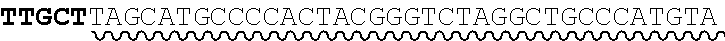 |
| NO: 1), which are located at the | 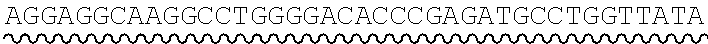 |
| end of the MHCK7 promoter | 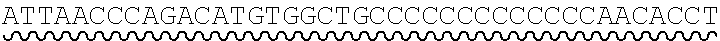 |
| sequence (i.e., at the -771 bp | 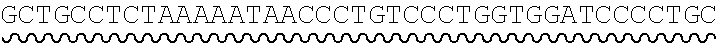 |
| position) and at the −581-−582 bp | 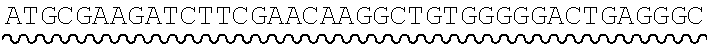 |
| position; and the underlined wavy | 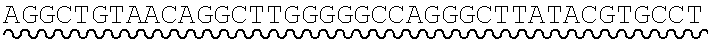 |
| line indicates the MHCK7 | 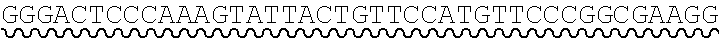 |
| promoter sequence.) | 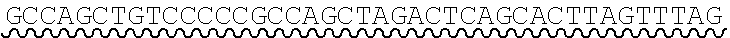 |
| | 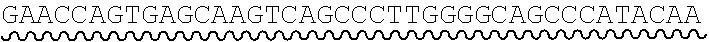 |
| | 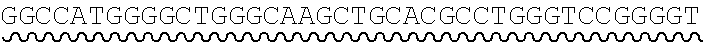 |
| | 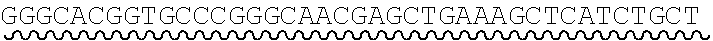 |
| | 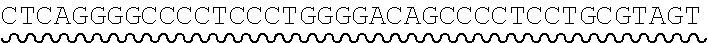 |
| | 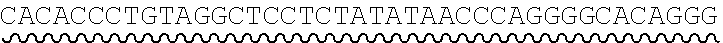 |
| | 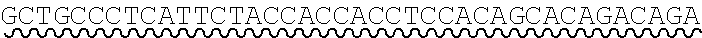 |
| | 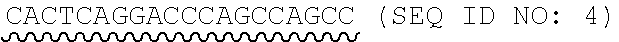 |
| Chimeric promoter 3 | GGCAGCTGTTGCTGGCAGCTGTTGCTGGCAGCTGTTG |
| (The sequences of recognition | 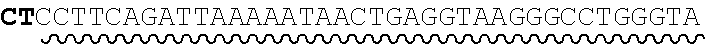 |
| and binding sites of the MyoD1 | 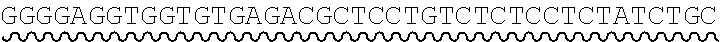 |
| transcription factor are shown in | 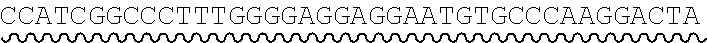 |
| bold, and the sequence contains | 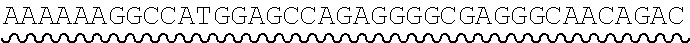 |
| three repeated recognition sites | 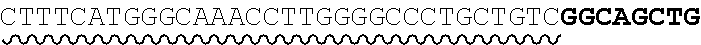 |
| of “GGCAGCTGTTGCT” (SEQ ID | 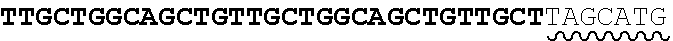 |
| NO: 1), which are located at the | 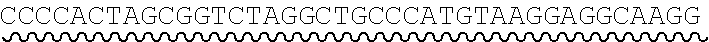 |
| end of the MHCK7 promoter | 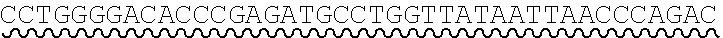 |
| sequence (i.e., −771 bp position), | 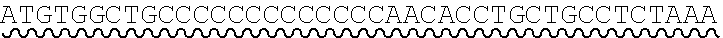 |
| −581-−582 bp position, and | 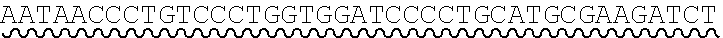 |
| −407-−406 bp position; and the | 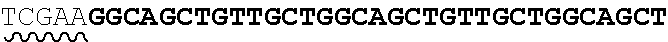 |
| underlined wavy line indicates the | 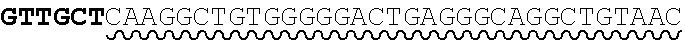 |
| MHCK7 promoter sequence) | 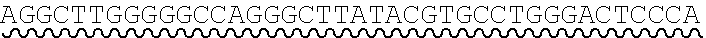 |
| | 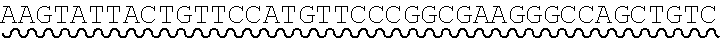 |
| | 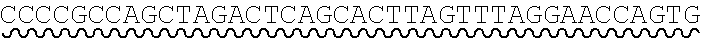 |
| | 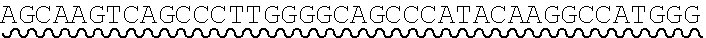 |
| | 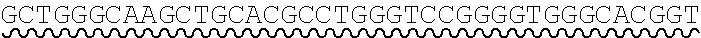 |
| | 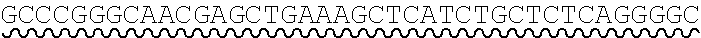 |
| | 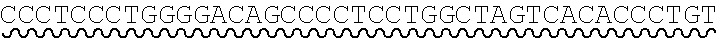 |
| | 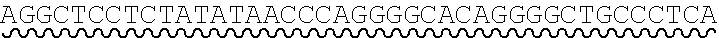 |
| | 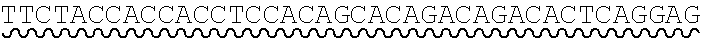 |
| | 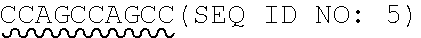 |

### Example 1 Construction of Plasmids

(1) Target plasmids were constructed using molecular tools such as seamless cloning. The primers were synthesized by Guangzhou Genewiz Biotechnology Co., Ltd. The following plasmids were synthesized:

| Number | Name | Use |
|---|---|---|
| 01 | pAAV.MHCK7.Fluc-2a- | Control plasmid, containing the |
| | maxGFP.WPRE.SV40pA | MHCK7 promoter |
| 02 | pAAV.MHCK7-1.Fluc-2a- | Experimental group plasmid 1, |
| | maxGFP.WPRE.SV40pA | containing the MHCK7 |
| | | promoter and two recognition |
| | | and binding sites of the MyoD1 |
| | | transcription factor (chimeric |
| | | promoter 1) |
| 03 | pAAV.MHCK7-2.Fluc-2a- | Experimental group plasmid 2, |
| | maxGFP.WPRE.SV40pA | containing the MHCK7 |
| | | promoter and four recognition |
| | | and binding sites of the MyoD1 |
| | | transcription factor (chimeric |
| | | promoter 2) |
| 04 | pAAV.MHCK7-3.Fluc-2a- | Experimental group plasmid 3, |
| | maxGFP.WPRE.SV40pA | containing the MHCK7 |
| | | promoter and nine recognition |
| | | and binding sites of the MyoD1 |
| | | transcription factor (chimeric |
| | | promoter 3) |

(2) First, plasmid No. 02 pAAV.MHCK7-1.Fluc-2a-maxGFP.WPRE.SV40pA was constructed. Plasmid No. 01 pAAV.MHCK7.Fluc-2a-maxGFP.WPRE.SV40pA was used as a template, which contained ITR sequences at both ends, the MHCK7 promoter (containing SEQ ID NO: 2), luciferase-p2A-maxGFP and other elements. Firstly, the high-fidelity enzyme PrimeSTAR® was used for amplification, and MHCK7-1-1-F1/MHCK7-1-1-R1, which carry one recognition binding site 5′-GGCAGCTGTTGCT-3′ (SEQ ID NO: 1) of the MyoD1 transcription factor respectively, were used as primers. The sequences are shown in Table 2. Plasmid No. 01 was used as a template to amplify the -582bp--771bp sequence of the MHCK7 promoter.

The reaction system was as follows:

| Ingredient | Volume |
|---|---|
| MHCK7-1-1-F1 (primer) | 1 μL |
| MHCK7-1-1-R1 (primer) | 1 μL |
| Plasmid No. 01 (template) | 50 ng |
| 2 *PrimeSTAR ® buffer | 25 μL |
| Deionized water | Added to 50 μL |

The amplification system:

| Temperature | Time | |
|---|---|---|
| 95° C. | 5 min for pre-denaturation | |
| 95° C. | 15 s for denaturation | 28 cycles |
| 58° C. | 15 s for annealing | |
| 72° C. | 30 s | |
| 72° C. | 5 min | |
| 4° C. | storage | |

The PCR products above were gel-recovered, 50 ng of gel-recovered products were taken as templates for the second amplification reaction, and primers MHCK7-1-1-F2/MHCK7-1-1-R1 were added. The sequences are shown in Table 2. A second PCR reaction was performed to extend the sequence of a terminal homology arm, and the reaction system and conditions were the same as above. The product of the second PCR reaction was subjected to gel recovery, and the recovered product was named fragment 1-1. At the same time, primers MHCK7-1-2-F1/MHCK7-1-2-R1 were used to amplify the MHCK7-1bp--581bp fragment with plasmid No. 01 as template, and gel recovery was performed. The recovered product was named fragment 1-2. Luciferase-p2A-maxGFP was amplified using Fluc-maxGFP-F1/Fluc-maxGFP-R1 as primers and plasmid No. 01 as template, and the recovered product was named fragment 1-3. The plasmid No. 01 was digested with MluI+EcoRI, and a 3356 bp fragment was recovered. The recovered product was named fragment 1-4.

(3) Seamless cloning was performed using Exnase Multis ligase to ligate the vector fragment to the PCR product; and the ligation system was as follows:

| Ingredient | Volume | |
|---|---|---|
| Fragment 1-1 | 50 | ng |
| Fragment 1-2 | 50 | ng |
| Fragment 1-3 | 50 | ng |
| Fragment 1-4 obtained by digesting the vector | 150 | ng |
| Exnase Multis ligase | 1 | μl |
| 5 × CE Multis buffer | 2 | μl |

| | |
|---|---|
| Deionized water | Added to 10 μl system |

A reaction at 37° C. for 30 min was sufficient.

(4) The ligation product was transfected into E. coli DH5α, and the transfection product was coated on an ampicillin plate; then the strains were picked for detection the next day, and the positive clones were sent to Guangzhou Genewiz Biotechnology Co., Ltd for sequencing. The plasmid containing the MHCK7 promoter with two recognition and binding sites of the MyoD1 transcription factor was selected. The plasmid with correct sequencing results was named pAAV.MHCK7-1.Fluc-2a-maxGFP.WPRE.SV40pA.

(5) Similarly, the-582bp--771bp sequence of the MHCK7 promoter was amplified using MHCK7-2-1-F1/MHCK7-2-1-R1, which carry 2 recognition and binding sites of the MyoD1 transcription factor respectively, as primers, and the plasmid No. 01 as a template for the PCR reaction. The PCR product was recovered by gel electrophoresis, and the PCR product as a template and primers MHCK7-2-1-F2/MHCK7-2-1-R1 were used for a second amplification. The fragment was recovered by gel electrophoresis and named as fragment 2-1. MHCK7-2-2-F1/MHCK7-1-2-R1 were used as primers and plasmid No. 01 as a template to amplify the -1bp--581bp fragment of the MHCK7 promoter, and the obtained product was named fragment 2-2. Luciferase-p2A-maxGFP was amplified using Fluc-maxGFP-F1/Fluc-maxGFP-R1 as primers and plasmid No. 01 as a template, and the recovered product was named fragment 2-3. Fragment 2-1, fragment 2-2, fragment 2-3, and fragment 1-4 obtained by enzymatic cleavage of the vector were ligated via seamless cloning. The plasmid containing the MHCK7 promoter with four recognition and binding sites of the MyoD1 transcription factor was selected. The plasmid with correct sequencing results was named pAAV.MHCK7-2.Fluc-2a-maxGFP.WPRE.SV40pA.

(6) In order to add a certain number of recognition and binding sites of the MyoD1 transcription factor at specific sequence positions, multiple amplifications were performed using primers carrying recognition and binding sites of the MyoD1 transcription factor. First, the first PCR reaction was performed using MHCK7-3-1-F1/MHCK7-3-1-R1 as primers and plasmid No. 01 as a template to amplify the-582bp--771bp sequence of the MHCK7 promoter such that the end of the sequence contained the recognition binding site of the MyoD1 transcription factor. The second amplification was performed using the first PCR product as a template and MHCK7-3-1-F2/MHCK7-3-1-R2 as primers, and the amplified product was recovered by gel electrophoresis. The third PCR reaction was performed using the second gel recovery product as a template and MHCK7-3-1-F3/MHCK7-3-1-R2 as primers. The PCR product was recovered by gel electrophoresis and the fragment was named 3-1. The -406--572 bp fragment of the MHCK7 promoter was amplified using MHCK7-3-2-F1/MHCK7-3-2-R1 as primers and plasmid No. 01 as a template, and the fragment was named 3-2. The -1--406 bp fragment of the MHCK7promoter was amplified using MHCK7-3-3-F1/MHCK7-1-2-R1 as primer and plasmid No. 01 as a template, and the fragment was named 3-3. Luciferase-p2A-maxGFP was amplified using Fluc-maxGFP-F1/Fluc-maxGFP-R1 as primers and plasmid No. 01 as a template, and the recovered product was named fragment 3-4. Fragment 3-1, fragment 3-2, fragment 3-3, fragment 3-4, and fragment 1-4 obtained by enzymatic cleavage of the vector were ligated via seamless cloning. The plasmid containing the MHCK7 promoter with nine recognition and binding sites of the MyoD1 transcription factor was selected. The plasmid with the correct sequencing results was named pAAV.MHCK7-3.Fluc-2a-maxGFP.WPRE.SV40pA.

TABLE 2
Primer sequences used in plasmid preparation

| Plasmid | | |
|---|---|---|
| number | Sequence name | Sequence (5′-3′) |
| Primer | MHCK7-1-1-F1 | ttcacgcgtGGCAGCTGTTGCTCCTTCAGATTAAAAATAACTGAGGTAA |
| list of | | GGGC (SEQ ID NO: 6) |
| plasmid | MHCK7-1-1-F2 | gcggccattcggtacaattcacgcgtGGCAGCTGTTGCTCCTTCAG |
| No. 02 | | (SEQ ID NO: 7) |
| | MHCK7-1-1-R1 | AAGCAACAGCTGCCGACAGCAGGGCCCCAAGGTTTGC (SEQ ID |
| | | NO: 8) |
| | MHCK7-1-2-F1 | GGCCCTGCTGTCGGCAGCTGTTGCTTAGCATGCCCCACTACGGGTC |
| | | TAGG (SEQ ID NO: 9) |
| | MHCK7-1-2-R1 | ggtggcgaccggtggatcggccgcGGCTGGCTGGCTCCTGAGT |
| | | (SEQ ID NO: 10) |
| | Fluc-maxGFP-F1 | Ggccgatccaccggtcgccaccatggaagatgccaaaaacattaagaag |
| | | (SEQ ID NO: 11) |
| | Fluc-maxGFP-R1 | ggttgattatctcgaGCGgaattcTCATCGAGCTCGAGATCTGGCG (SEQ ID NO: 12) |
| Primer | MHCK7-2-1-F1 | gtGGCAGCTGTTGCTGGCAGCTGTTGCTCCTTCAGATTAAAAATAAC |
| list of | | TGAGGTAAGGG (SEQ ID NO: 13) |
| plasmid | MHCK7-2-1-F2 | ccattcggtacaattcacgcgtGGCAGCTGTTGCTGGCAGCTGTTGCTCCTTCA |
| No. 03 | | GATT (SEQ ID NO: 14) |
| | MHCK7-2-1-R1 | GGGGCATGCTAAGCAACAGCTGCCAGCAACAGCTGCCGACAGCA |
| | | GGGCCCCAAGGTTTG (SEQ ID NO: 15) |
| | MHCK7-2-2-F1 | GTCGGCAGCTGTTGCTGGCAGCTGTTGCTTAGCATGCCCCACTACG |
| | | GGTCTAGGC (SEQ ID NO: 16) |
| | MHCK7-1-2-R1 | ggtggcgaccggtggatcggccgcGGCTGGCTGGCTCCTGAGT |
| | | (Same as above, SEQ ID NO: 10) |
| | Fluc-maxGFP-F1 | Ggccgatccaccggtcgccaccatggaagatgccaaaaacattaagaag (Same |
| | | as above, SEQ ID NO: 11) |
| | Fluc-maxGFP-R1 | ggttgattatctcgaGCGgaattcTCATCGAGCTCGAGATCTGGCG (Same as |
| | | above, SEQ ID NO: 12) |
| Primer | MHCK7-3-1-F1 | CTGTTGCTGGCAGCTGTTGCTCCTTCAGATTAAAAATAACTGAGGT |
| list of | | AAGGGCCTGGG (SEQ ID NO: 17) |
| plasmid | MHCK7-3-1-F2 | cattcggtacaattcacgcgtGGCAGCTGTTGCTGGCAGCTGTTGCTGGCAGCT |
| No. 04 | | GTTGCTCCTTCAG (SEQ ID NO: 18) |
| | MHCK7-3-1-F3 | gttcctgcggccattcggtacaattcacgcgtG (SEQ ID NO: 19) |
| | MHCK7-3-1-R1 | CAACAGCTGCCAGCAACAGCTGCCAGCAACAGCTGCCGACAGCA |
| | | GGGCCCCAAGGTTTG (SEQ ID NO: 20) |
| | MHCK7-3-1-R2 | TGGGGCATGCTAAGCAACAGCTGCCAGCAACAGCTGCCAGCAACA |
| | | GCTGCCGACAGCAG (SEQ ID NO: 21) |
| | MHCK7-3-2-F1 | GCAGCTGTTGCTTAGCATGCCCCACTACGGGTCTAGGCTG (SEQ ID |
| | | NO: 22) |
| | MHCK7-3-2-R1 | GCAACAGCTGCCAGCAACAGCTGCCAGCAACAGCTGCCTTCGAAG |
| | | ATCTTCGCATGCAG (SEQ ID NO: 23) |
| | MHCK7-3-3-F1 | GGCAGCTGTTGCTGGCAGCTGTTGCTGGCAGCTGTTGCTCAAGGC |
| | | TGTGGGGGACTGAG (SEQ ID NO: 24) |
| | MHCK7-1-2-R1 | ggtggcgaccggtggatcggccgcGGCTGGCTGGCTCCTGAGT (Same as above, |
| | | SEQ ID NO: 10) |
| | Fluc-maxGFP-F1 | Ggccgatccaccggtcgccaccatggaagatgccaaaaacattaagaag (Same |
| | | as above, SEQ ID NO: 11) |
| | Fluc-maxGFP-R1 | ggttgattatctcgaGCGgaattcTCATCGAGCTCGAGATCTGGCG |
| | | (Same as above, SEQ ID NO: 12) |

### Example 2 Preparation of Recombinant Adeno-Associated Viruses

To better validate the tissue specificity and expression activity of the optimized MHCK7 promoter, adeno-associated virus serotype 9 (AAV9) was packaged and transfected into mice, followed by measurement of expression efficiency of the luciferase protein for validation.

Packaging of recombinant adeno-associated viruses: Cells (HEK293 cells) were inoculated at a density of 5E+5 (5×10⁵) cells/ml in a 15 cm culture dish and incubated overnight for 16-18 hours. 15 μg of pHelper, 10 μg of pRep2Cap9, and 7 μg of plasmid No. 01 pAAV.MHCK7.Fluc-2a-maxGFP.WPRE.SV40pA (plasmid No. 02, plasmid No. 03, or plasmid No. 04), and 10 μg of the transfection reagent polyethyleneimine were added to each culture dish for incubation and transfection. After 72 hours of transfection, cells and supernatant were collected and centrifuged through iodixanol density gradient. The virus titer was determined by SYBR™ Green I qPCR and stored in a −80° C. refrigerator before use.

### Example 3 In vivo Imaging and Tissue Sampling of Mice Injected with Viruses

Twenty 5-6 week old BALB/c mice were randomly divided into 4 groups, 5 mice in each group. The groups were set as follows:

| Group | Name | Use |
|---|---|---|
| Group | pAAV.MHCK7.Fluc-2a- | Mice injected with control |
| A | maxGFP.WPRE.SV40pA | viruses containing the MHCK7 |
| | | promoter |
| Group | pAAV.MHCK7-1.Fluc-2a- | The first group of mice injected |
| B | maxGFP.WPRE.SV40pA | with the experimental viruses, |
| | | which contain the MHCK7 |
| | | promoter and two recognition |
| | | and binding sites of the MyoD1 |
| | | transcription factor (the chimeric |
| | | promoter 1) |
| Group | pAAV.MHCK7-2.Fluc-2a- | The second group of mice |
| C | maxGFP.WPRE.SV40pA | injected with the experimental |
| | | viruses, which contain the |
| | | MHCK7 promoter and four |
| | | recognition and binding sites of |
| | | the MyoD1 transcription factor |
| | | (the chimeric promoter 2) |
| Group | pAAV.MHCK7-3.Fluc-2a- | The third group of mice injected |
| D | maxGFP.WPRE.SV40pA | with the experimental viruses, |
| | | which contain the MHCK7 |
| | | promoter and nine recognition |
| | | and binding sites of the MyoD1 |
| | | transcription factor (the chimeric |
| | | promoter 3) |

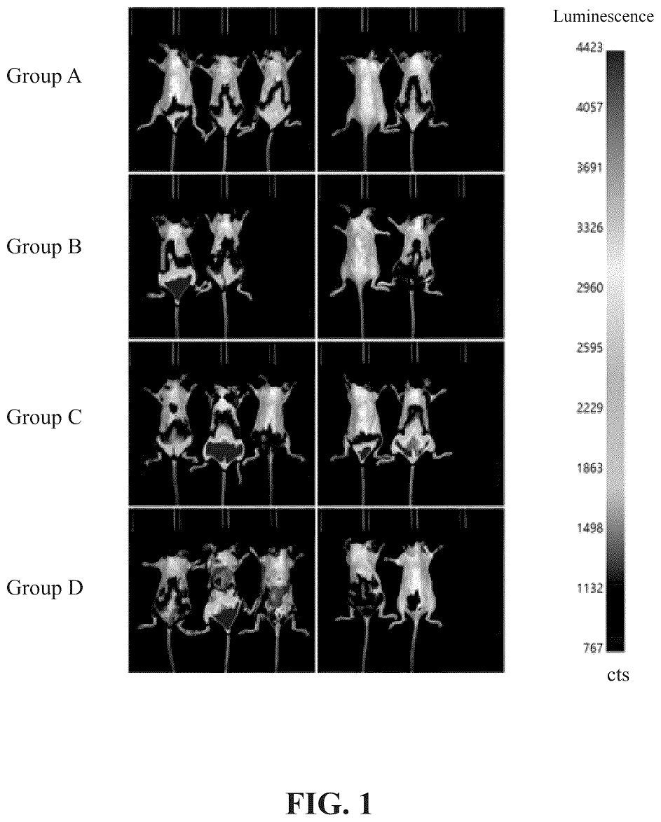

The above viruses (titer: 2.5×1012 GC/mL, injection volume: 200 μL) was injected into the tail vein of a mouse. The day of virus injection was defined as day 1, and in vivo imaging of mice was performed 21 days later. The results are shown in FIG. 1. As shown in FIG. 1, compared with the control group A, the expression of luciferase in groups B, C, and D is enhanced to varying degrees. Tissue sampling of mice was performed after 22 days. Six tissue sites were collected, namely, liver, biceps, quadriceps, heart, brain, and lung. The collected tissue samples were stored in a −80° C. refrigerator.

### Example 4 RNA Extraction of Mouse Tissue Samples and Quantification of RNA by Reverse Transcription

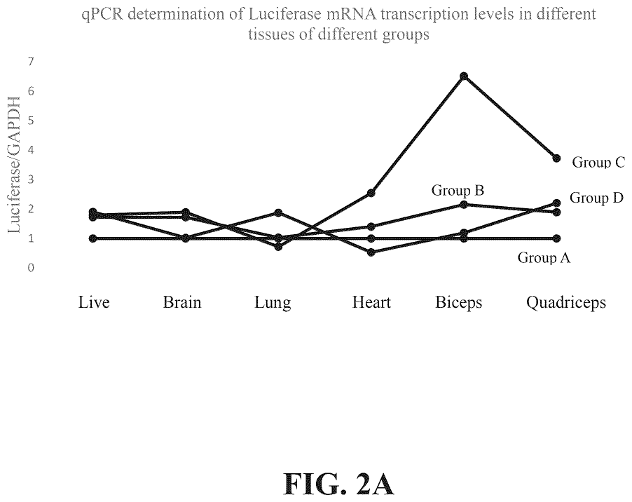

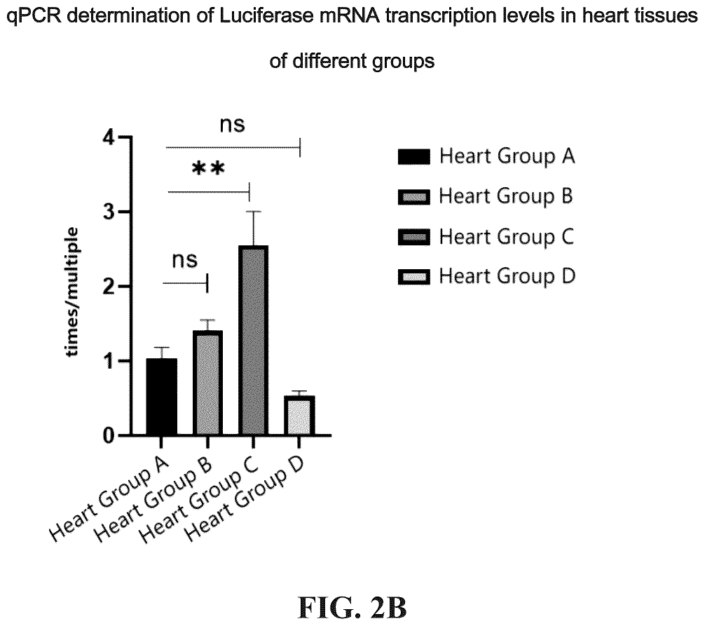

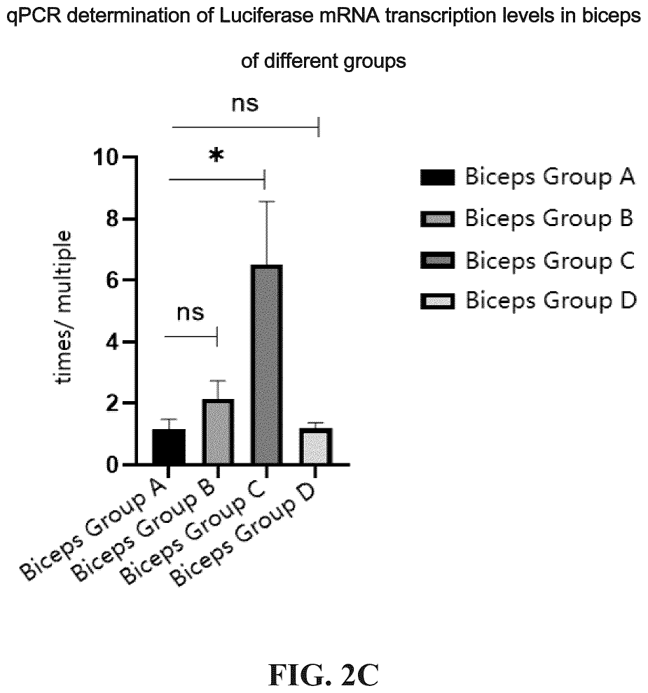

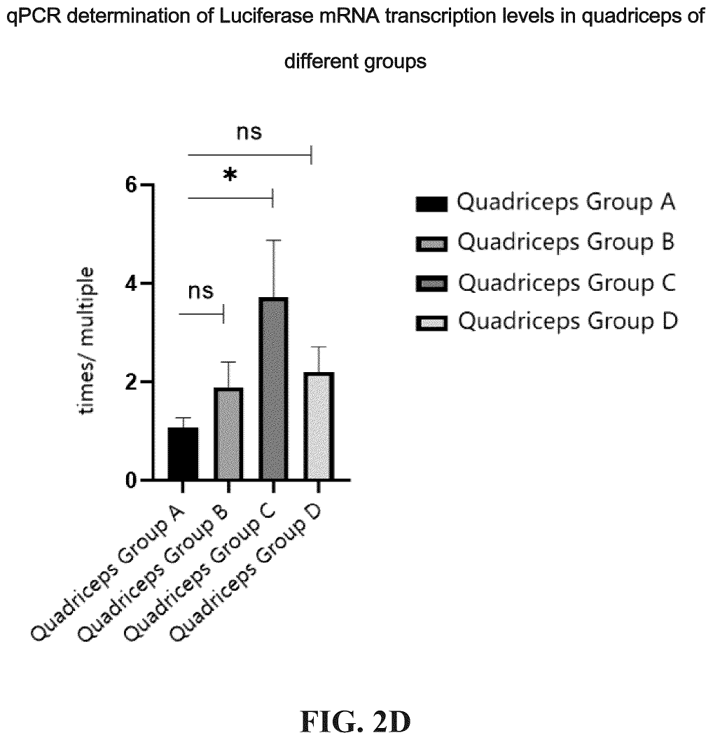

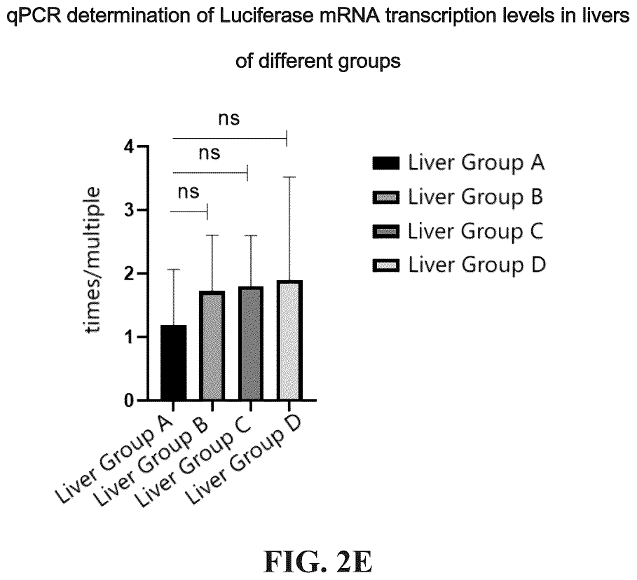

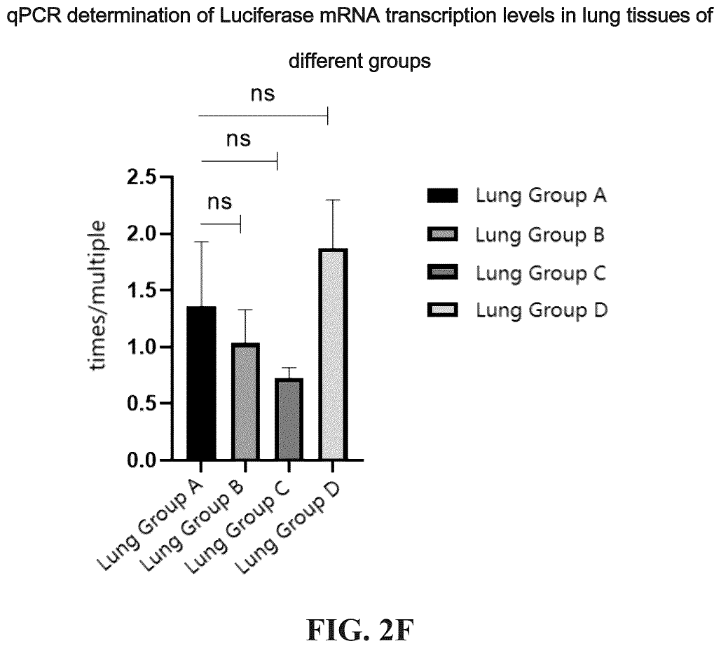

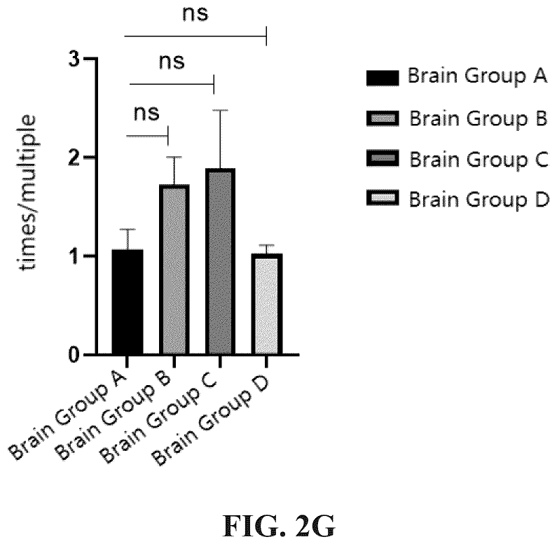

The operation was performed according to the step-by-step instructions of the RNA extraction kit. 0.1-0.5 g of a tissue sample was taken and transferred to a 1.5 ml EP tube containing 1 ml of transzol up, to which two RNase free steel beads were added. Grinding was performed using a shaking homogenizer at 70 Hz, with oscillation for 50 seconds and rest for 10 seconds, and this operation was repeated 7 times. The EP tube was allowed to stand at room temperature for 5 min, centrifuged, and the supernatant was collected. Chloroform was added to the collected supernatant at a volume ratio of 5:1, and the mixture was shaken vigorously for 30 seconds. The resulting mixed solution was allowed to stand at room temperature for 3 minutes. The obtained mixed solution was centrifuged at 4° C. The supernatant obtained by centrifugation was collected, an equal volume of anhydrous ethanol was added, and lightly mixed. The resulting solution was added to a spin column, centrifuged, and the waste liquid was discarded. 500 μl of CB9 was added to the obtained spin column, and the spin column was centrifuged at room temperature to discard the waste liquid; this operation was repeated twice. 500 μl of WB9 was added to the obtained spin column, and the spin column was centrifuged at room temperature and the waste liquid was discarded; this was repeated twice. The obtained spin column was centrifuged without load for 20 seconds at room temperature to remove residual ethanol. 50 μl of RNase-free water was added to the resulting spin column for elution. The concentration of the extracted RNA was measured using NanoDrop™. 500 ng RNA was taken for reverse transcription. SYBR™ Green I qPCR was used for quantification, and the results are shown in FIGS. 2A, 2B, 2C, 2D, 2E, 2F, 2G, and Table 3. Compared with the control group A, the expression of luciferase in different muscle tissues of group C, including heart, biceps and quadriceps, is increased, with the average increase in biceps being 6.51 times and in quadriceps being 3.72 times; at the same time, there was no significant difference in the increase of luciferase expression in non-muscle tissues such as liver, lung, and brain. This indicates that the MHCK7 promoter optimized in group C has good results.

TABLE 3
Results of qPCR assay
qPCR determination of luciferase mRNA transcription
levels in different tissues of different groups

| | average growth multiple | | | | | | |
|---|---|---|---|---|---|---|---|
| Group | Live | Brain | Lung | Heart | Biceps | Quadriceps | note |
| Group A | 1 | 1 | 1 | 1 | 1 | 1 | control group |
| Group B | 1.72 | 1.72 | 1.03 | 1.4 | 2.15 | 1.89 | experimental |
| | | | | | | | group |
| Group C | 1.79 | 1.89 | 0.72 | 2.54 | 6.51 | 3.72 | experimental |
| | | | | | | | group |
| Group D | 1.9 | 1.02 | 1.87 | 0.53 | 1.19 | 2.2 | experimental |
| | | | | | | | group |

### Example 5 Western Blot Experiments of Mouse Tissue Samples

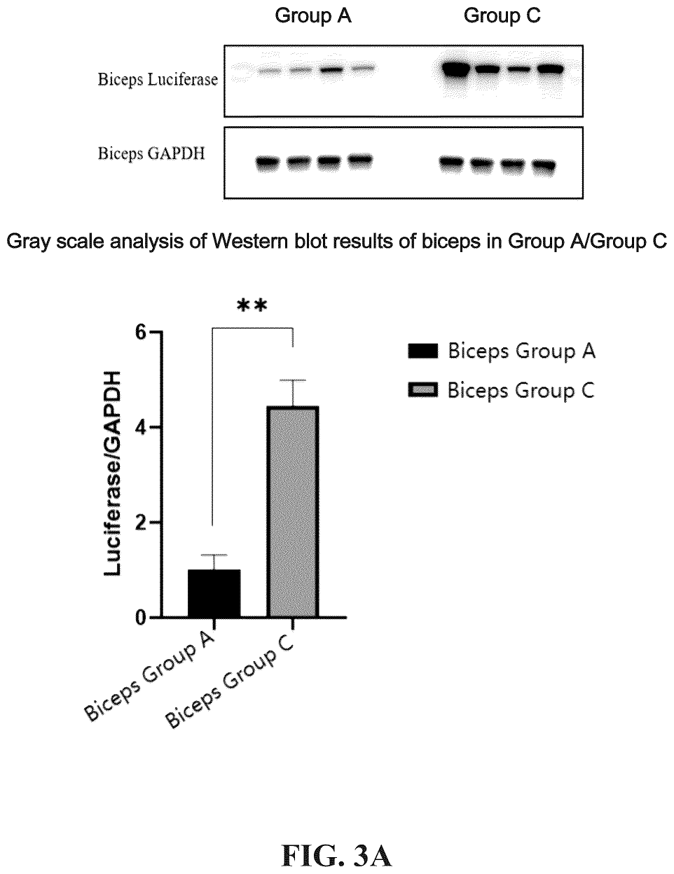

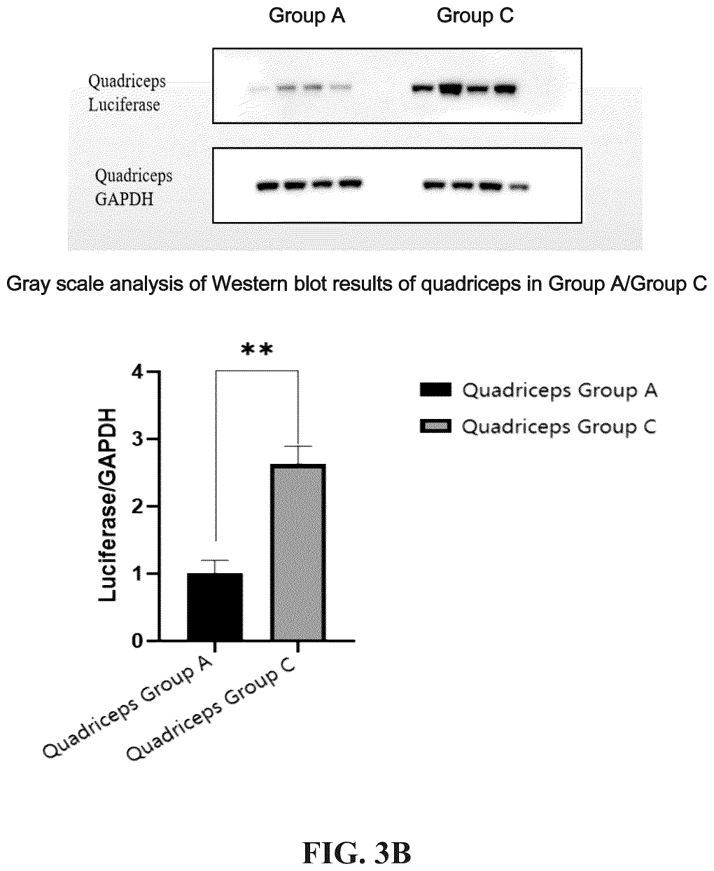

The biceps and quadriceps of 4 mice were randomly taken from group A and group C for a Western blot analysis. The method was as follows: The tissue was sheared into fine fragments and 250 μl of RIPA lysate containing a final concentration of 1 mM phenylmethanesulfonyl fluoride (PMSF) was added. Two sterilized zirconium oxide grinding balls were added to the resulting lysis product, and then the product was balanced and placed in a pre-cooled grinder for grinding. The grinding procedure was: The grinding temperature was −20° C., the grinding frequency was 70 Hz, each oscillation was 50 seconds, followed by a pause for 20 seconds, and this operation was repeated 5-7 times. After grinding, the ground product was centrifuged at 12000 rpm and 4° C. for 15 min. The supernatant of the centrifuged product was carefully aspirated and the protein concentration was determined using a BCA protein assay kit. 20 μg of protein samples were taken separately for running on a SDS-PAGE gel. The results are shown in FIG. 3A and FIG. 3B. The Western blot analysis shows that compared with control group A, the expression of luciferase protein in the biceps of group C increases by 4.45 times, and the expression of luciferase protein in the quadriceps increases by 2.63 times, and the enhancement effect is very significant.

The above embodiments are preferred embodiments of the present disclosure, but the embodiments of the present disclosure are not limited to the above embodiments. Any other changes, modifications, substitutions, combinations, and simplifications that do not deviate from the spirit and principles of the present disclosure shall be considered as equivalent replacement methods and shall be included in the scope of the present disclosure.

### References

[1] Maja Z Salva, Charis L Himeda, Phillip WI Tai, et al. Design of tissue-specific regulatory cassettes for high-level rAAV-mediated expression in skeletal and cardiac muscle [J]. Mol Ther, 2007, 15(2):320-329.
[2] Sun B, Young S P, Li P, et al. Correction of Multiple Striated Muscles in Murine Pompe Disease Through Adeno-associated Virus-mediated Gene Therapy [J]. Molecular Therapy: the Journal of the American Society of Gene Therapy, 2008, 16(8):1366-1371.
[3] Blum R, Dynlacht B D. The role of MyoD1 and histone modifications in the activation of muscle enhancers [J]. Epigenetics: Official Journal of the DNA Methylation Society, 2013, 8(8):778-784.

1. A muscle-specific chimeric promoter, comprising:
(1) a hybrid α-myosin heavy chain enhancer/muscle creatine kinase enhancer-promoter (MHCK7 promoter); and
(2) one or more binding sites of a transcription factor,
wherein the MHCK7 promoter comprises the sequence set forth in SEQ ID NO: 2 or a functional variant thereof having at least 90% sequence identity to SEQ ID NO: 2, and
wherein the transcription factor is a member selected from the group consisting of the MyoD family of transcription factors.

2. The muscle-specific chimeric promoter according to claim 1, wherein the transcription factor is MyoD1 and/or Myog.

3. The muscle-specific chimeric promoter according to claim 1, wherein a number of the one or more binding sites of the transcription factor is 2-9.

4. The muscle-specific chimeric promoter according to claim 1, wherein a number of the one or more binding sites of the transcription factor is 2, 4, or 9.

5. The muscle-specific chimeric promoter according to claim 1, wherein the one or more binding sites of the transcription factor comprise the sequence set forth in SEQ ID NO: 1, or a functional variant comprising 1 or 2 nucleotide changes when compared to the sequence set forth in SEQ ID NO: 1.

6. The muscle-specific chimeric promoter according to claim 1, wherein the one or more binding sites of the transcription factor are located upstream and/or downstream of the MHCK7 promoter and/or located within the MHCK7 promoter.

7. The muscle-specific chimeric promoter according to claim 1, comprising the sequence set forth in SEQ ID NO: 3, SEQ ID NO: 4, or SEQ ID NO: 5, or a functional variant having 90% sequence identity to any one of SEQ ID NOs: 3-5.

8. The muscle-specific chimeric promoter according to claim 1 which has a higher capacity to initiate transcription in muscle tissue when compared to the MHCK7 promoter.

9. The muscle-specific chimeric promoter according to claim 8, wherein the muscle tissue is selected from the group consisting of skeletal muscle and cardiac muscle;
preferably, the skeletal muscle is a bicep and/or a quadricep.

10. A gene expression cassette comprising the muscle-specific chimeric promoter of claim 1 and a target gene operably linked to the muscle-specific chimeric promoter.

11. An expression vector comprising the muscle-specific chimeric promoter of claim 1 or a gene expression cassette, wherein the gene expression cassette comprises the muscle-specific chimeric promoter of claim 1 and a target gene operably linked to the muscle-specific chimeric promoter.

12. The expression vector according to claim 11, which is a viral expression vector.

13. The expression vector according to claim 11, which is an expression vector of an adeno-associated virus (AAV).

14. A host cell comprising the muscle-specific chimeric promoter of claim 1, a gene expression cassette, or an expression vector,
wherein the gene expression cassette comprises the muscle-specific chimeric promoter of claim 1 and a target gene operably linked to the muscle-specific chimeric promoter, and
wherein the expression vector comprises the muscle-specific chimeric promoter of claim 1 or the gene expression cassette.

15. A pharmaceutical composition, comprising:
(1) a gene expression cassette comprising the muscle-specific chimeric promoter of claim 1 and a target gene operably linked to the muscle-specific chimeric promoter;
an expression vector comprising the muscle-specific chimeric promoter of claim 1 or the gene expression cassette; or
a host cell comprising the muscle-specific chimeric promoter of claim 1, the gene expression cassette, or the expression vector; and
(2) a pharmaceutically acceptable carrier.

16. A method of treating a muscle tissue-related disease, comprising administering to a subject in need an effective amount of a gene expression cassette, an expression vector, a host cell, or a pharmaceutical composition,
wherein the gene expression cassette comprises the muscle-specific chimeric promoter of claim 1 and a target gene operably linked to the muscle-specific chimeric promoter,
wherein the expression vector comprises the muscle-specific chimeric promoter of claim 1 or the gene expression cassette,
wherein the host cell comprises the muscle-specific chimeric promoter of claim 1, the gene expression cassette, or the expression vector, and
wherein the pharmaceutical composition comprises (1) the muscle-specific chimeric promoter of claim 1, the gene expression cassette, the expression vector, or the host cell; and (2) a pharmaceutically acceptable carrier.

17. The muscle-specific chimeric promoter according to claim 2, wherein a number of the one or more binding sites of the transcription factor is 2-9.

18. The muscle-specific chimeric promoter according to claim 3, wherein the one or more binding sites of the transcription factor comprise the sequence set forth in SEQ ID NO: 1, or a functional variant comprising 1 or 2 nucleotide changes when compared to the sequence set forth in SEQ ID NO: 1.

19. The muscle-specific chimeric promoter according to claim 18, wherein the number of the one or more binding sites of the transcription factor is 2, 4, or 9.

20. The muscle-specific chimeric promoter according to claim 19, wherein the one or more binding sites of the transcription factor are located upstream and/or downstream of the MHCK7 promoter and/or located within the MHCK7 promoter.
